(19) United States
(12) Patent Application Publication
(10) Pub. No.: US 2006/0106395 A1
(43) Pub. Date: May 18, 2006

(54) Instrument set and method for working a cervical vertebral body

(75) Inventors: Helmut D. Link, Hamburg (DE); Paul C. McAfee, Baltimore, MD (US)

Correspondence Address: Barry E. Bretschneider;Morrison & Foerster LLP, Suite 300, 1650 Tysons Boulevard, McLean, VA, 22102, US

(73) Assignee: Cervitech, Inc., Rockaway, NJ (US)

(21) Appl. No.: 10/897,404

(22) Filed: Nov. 12, 2004

(51) Int. Cl. A61B 17/90; A61F 2/44; A61F 2/46

(52) U.S. Cl. 606/87; 623/17.11

(57) ABSTRACT

The instrument set and the method for preparing a cervical vertebral body (2) to receive an abutment surface (13) of an intervertebral joint prosthesis (11) in the intervertebral space (1) are characterized in that a guide element (3), which is to be inserted into the intervertebral space (1), and a working instrument (10), for working at least one vertebral body (2), have interacting guide surfaces (5, 9a). The working range of the working instrument is limited by the guide surfaces (5, 9a).

The invention relates to an instrument set and a method for working a cervical vertebral body to receive an intervertebral joint prosthesis in the intervertebral space.

When fitting an intervertebral joint prosthesis as a replacement for an intervertebral disk, the operating surgeon is working in an operating site which is very difficult to see and which is in immediate proximity to important nerve paths and blood vessels. This applies in particular to the area of the cervical spine, because in this area there is particularly little distance between the parts of the vertebral bone to be worked and sensitive adjoining areas. Therefore, as regards instruments which have a particular potential for causing damage or which have to be inserted with particular precision, it is sought to limit their freedom of movement to the necessary extent by means of suitable instruments.

An instrument set is already known (WO 03/075774 A1) for preparing an intervertebral space to receive an intervertebral joint prosthesis, the freedom of movement of a working instrument being limited by guide surfaces of a fixing and guiding device. With the aid of an adjustment instrument inserted between the vertebral bodies, the fixing and guiding device is brought to a defined position relative to the vertebrae and is screwed securely to the vertebrae.

After removal of the adjustment instrument, the intervertebral space can be worked to the extent defined by the fixing and guiding device. A disadvantage of this instrument set is that quite a few preparatory steps are needed before it can be used to work the intervertebral space. Moreover, the described instrument set is not suitable for working the ventral area of the vertebral body, because the fixing and guiding device secured to the vertebrae obstructs access.

The object of the invention is to make available an instrument set and a method for working the ventral area of the vertebral body.

The solution according to the invention lies in the features of the claims. The basic concept of the method is that a guide element is positioned in the intervertebral space and has a guide surface, and that the vertebral body is worked by means of a working instrument while the latter is guided on the guide surface. The instrument set comprises a guide element, to be inserted into the intervertebral space, and a working instrument, for working at least one vertebral body, which have interacting guide surfaces.

To insert an intervertebral joint prosthesis, the vertebrae concerned are first of all accessed from the ventral direction, and the intervertebral space is freed from the intervertebral disk. The guide element according to the invention is inserted into the free space thus created. In the inserted state, the guide element is in contact at different places with the vertebral body surfaces adjoining the intervertebral space. By virtue of this contact, the guide element automatically assumes a defined position inside the intervertebral space. The areas of the guide element which bear on the vertebral body surfaces act as positioning surfaces.

In this way, the guide element is situated in a defined position with respect to both vertebrae concerned, and it can be used as a fixed point for guiding the working instrument with which the vertebral body is worked. The guide element comprises guide surfaces which cooperate with guide surfaces of the working instrument and on which the working instrument is guided during working.

The position of the vertebrae with respect to one another in the healthy state is determined by the interplay of intervertebral disk and ligaments. After removal of the intervertebral disk which has been holding the vertebrae apart, the ligaments draw the vertebrae together. Their tensioning ensures that the guide element located between two vertebrae is fixed by clamping. The thickness of the guide element is dimensioned so as to bring about this clamping.

To receive the joint prosthesis, the intervertebral space is prepared so as to match the outer shape of the prosthesis. In an advantageous embodiment, the instrument set according to the invention is put to use after the intervertebral space has been so prepared, and the guide element has an outer shape which corresponds to the prosthesis such that both are inserted into the intervertebral space. The guide element has a ventro-dorsal depth which is normally not greater than the depth of that part of the prosthesis lying behind the flanges. After the guide element has been inserted into the intervertebral space, the guide surface thus lies deep in the intervertebral space. Because of this matching shape and the thereby defined position of the guide element with respect to the vertebrae, the further working of the vertebral bodies carried out with the aid of the guide element can be adapted exactly to the subsequent position of the prosthesis. It is not absolutely essential that the outer shape of the guide element inside the intervertebral space corresponds completely to the prosthesis shape. Instead, a partial correspondence is sufficient in which the positioning surfaces of the guide element represent parts of the surface shape of the prosthesis.

In an advantageous embodiment, the guide surface of the working instrument is movable in the lateral direction with respect to the guide surface of the guide element. Only the position of the working instrument in the cranio-caudal direction is then fixed. The working instrument can be moved freely perpendicular thereto. During working, the instrument is guided on the guide surface parallel to the surface which is to be created by the working.

The guide surface of the working instrument is preferably a guide mandrel. The working instrument is also preferably a cylindrical milling cutter, and the guide mandrel is rotatable relative to the guide surfaces of the guide element. The working range of the cylindrical milling cutter is then defined on both sides of the guide surfaces of the guide element by the diameter of the cylindrical milling cutter. This diameter is preferably between 5 and 10 mm.

In another advantageous embodiment, the working instrument is a hand tool, for example a curette or a rasp.

The guide surface of the guide element is advantageously arranged centrally between the vertebral bodies. In this way, the vertebral bodies above and below the guide element can be worked simultaneously in one procedure. However, it is also possible to work on just one side if the working instrument extends on only one side from the guide surface, or if the guide surface is arranged eccentrically.

To permit safe insertion of the guide element despite the difficulty in viewing the operating site, the instrument set preferably comprises an insertion element which can be gripped in order to maneuver the guide element. The insertion element is also preferably connected to the guide element before insertion and is again removed from it after insertion.

The secure hold of the guide element in the intervertebral space is advantageously supported by the surface configuration of the positioning surfaces. To this end, projections for mechanical engagement in the surface of the vertebral body can be provided on the positioning surfaces. These prevent a movement of the guide element relative to the vertebral bodies even when forces are applied to the guide element by the working instrument during working. The projections can, for example, be configured as teeth, the tips of said teeth being directed toward the vertebral body surfaces, or a surface roughness can be created by regularly or irregularly distributed smaller projections.

The invention is described below on the basis of an advantageous illustrative embodiment and with reference to the attached drawings, in which:

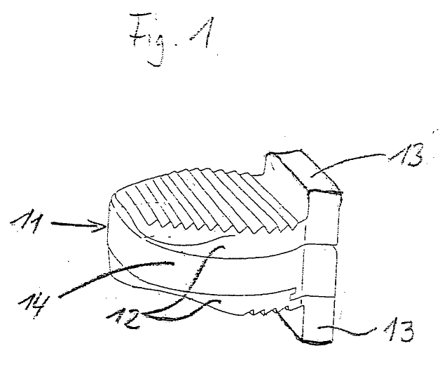

FIG. 1 shows a perspective top view of an intervertebral joint prosthesis;

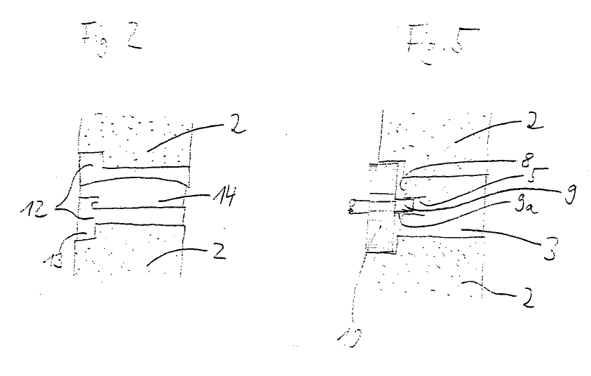

FIG. 2 shows a sagittal section through an intervertebral joint prosthesis inserted into the intervertebral space;

FIG. 3 shows a guide element according to the invention with an insertion element prior to insertion into the intervertebral space;

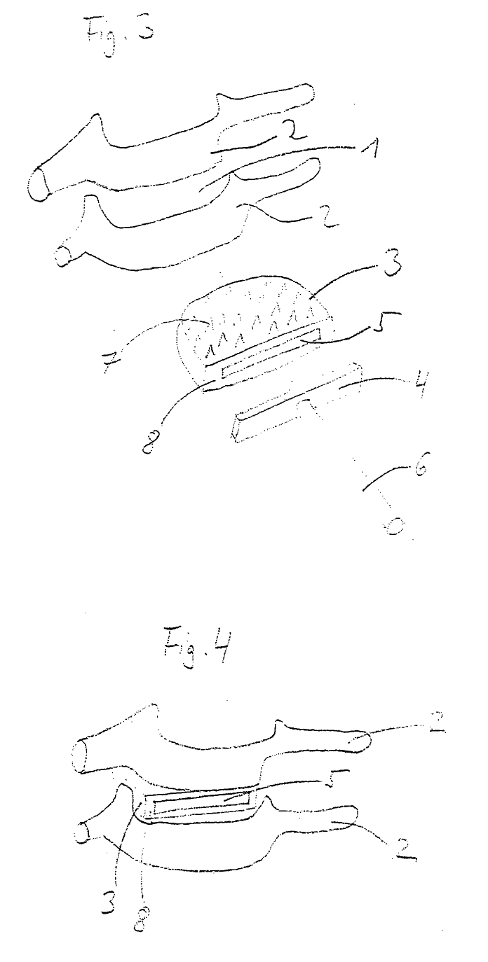

FIG. 4 shows a guide element according to the invention inserted into the intervertebral space; and

FIG. 5 shows a sagittal section through a guide element inserted into the intervertebral space, with the working instrument according to the invention.

The intervertebral joint prosthesis 11 shown in FIG. 1 consists of endplates 12 and of a prosthesis core 14 and forms a joint for replacement of the intervertebral disk. Each endplate 12, or at least one endplate 12, has, at the ventral margin, a flange 13 which rises in the manner of a ledge above the surface of the endplate 12 to be connected to the vertebral body. As is shown in the cross-sectional view in FIG. 2, the prosthesis is intended to be inserted into an intervertebral space 1 between vertebral bodies 2. In order to avoid irritation of surrounding organs by ventrally protruding parts of the prosthesis 11, the flanges 13 are recessed into the vertebral bodies 2. For this purpose, before the prosthesis 11 is inserted, material is removed from the vertebral bodies 2 such that the flanges 13 are accommodated completely or for the most part therein and thus do not protrude ventrally beyond the original contour of the vertebral body 2 or do so only slightly. The instrument set according to the invention is used to work the vertebral bodies 2 in the manner necessary for this recessing.

Before the instrument set according to the invention is fitted, the intervertebral disk is removed. Although not absolutely necessary, the surface of the vertebral bodies is generally prepared for receiving the prosthesis 11. For this purpose, the surfaces of the vertebral bodies 2 directed toward the intervertebral space 1 are worked in such a way that the intervertebral space 1 has a shape matching the prosthesis 11 as far as possible. Working of the ventral margin of the vertebral body for receiving the flange 13 is thus still to be done. This working is carried out with the aid of the guide element 3 to be inserted into the intervertebral space 1. To ensure that the vertebral bodies 2, during working, are positioned relative to one another exactly as is to be expected after insertion of the prosthesis, the guide element 3, insofar as it comes into contact with the vertebral bodies 2 in the intervertebral space 1, has a shape corresponding to the prosthesis 11. It is not absolutely essential that the shape of the guide element 3 corresponds completely to the shape of the prosthesis 11. Instead, it suffices if the positioning faces forming the surface of the guide element 3 represent corresponding parts of the prosthesis 11 or correspond to these.

To insert the guide element 3 into the intervertebral space 1, an insertion element 4 is connected in a suitable manner (not shown). The grip part 6 of the insertion element 4 is taken hold of for maneuvering the guide element 3 connected to the insertion element 4. The two vertebral bodies 2 are spread (in a manner not shown) counter to the tensile force of the ligaments, so that the intervertebral space 1 is sufficiently large for receiving the guide element 3. However, the guide element 3 can also be pressed into the intervertebral space as it spreads the vertebral bodies 2 apart. After insertion of the guide element 3 into the intervertebral space 1, the ligaments fix the guide element 3 by clamping. The secure hold of the guide element 3 is supported by projections 7 on the surface of the guide element which engage in the surfaces of the vertebral bodies 2 adjacent to the intervertebral space 1. The projections 7 can be configured in different ways. For example, they can be in the form of teeth, or smaller projections can be used to create a surface roughness. The insertion element 4 is released from the guide element 3 fixed in the clamped position.

The guide element 3 is located in the intervertebral space 1 in the manner illustrated in FIG. 4. Only a front face 8, with a groove forming the guide surface 5, is still accessible to the operating surgeon.

As is shown in FIG. 5, a guide surface 9a of a cylindrical milling cutter 10 cooperates with the guide surface 5 of the guide element 3. The guide surface 9a of the cylindrical milling cutter 10 is here the outer surface of a guide mandrel 9. Instead of the cylindrical milling cutter 10, the working instrument can also be a hand tool such as a rasp or curette with a corresponding guide mandrel 9.

The cylindrical milling cutter 10 expediently has a diameter of between 5 and 10 mm, rotates together with the guide mandrel 9 and in so doing removes material from the vertebral bodies 2. The guide surface 5 of the guide element 3 is arranged centrally between the vertebral bodies 2 so that both vertebral bodies 2 are worked simultaneously. The cylindrical milling cutter 10 can be moved in the lateral direction inside the guide surface 5, its position in the cranio-caudal direction being defined by the guide surface 5. Its working depth in the same direction is defined by its diameter. It is at least equal to the cranio-caudal dimension of the prosthesis at the flanges. The guide surface 5 also defines the depth of penetration of the milling cutter into the vertebral bodies 2 in the ventro-dorsal direction. The maximum depth of penetration is reached when the guide mandrel 9 strikes the bottom of the guide face 5 or when the milling cutter bears on the front face 8 of the guide element 3.

Once the working of the vertebral bodies 2 has been completed, the guide element 3 is removed again from the intervertebral space 1. To do this, the guide element 3 is connected to the insertion element 4 and pulled out of the intervertebral space 1. The prosthesis 11 can then be inserted.

1. Instrument set for working a cervical vertebral body to receive an intervertebral joint prosthesis in the intervertebral space, characterized in that a guide element (3), which is to be inserted into the intervertebral space (1), and an instrument (10), for working at least one vertebral body, have interacting guide surfaces (5, 9a).

2. Instrument set according to claim 1, characterized in that the guide element (3) has positioning surfaces which interact with the vertebral body surfaces adjoining the intervertebral space (1).

3. Instrument set according to claim 2, characterized in that the positioning surfaces of the guide element (3) represent the surface shape of the prosthesis (11) or parts thereof.

4. Instrument set according to claim 1, characterized in that the thickness of the guide element (3) is sufficient to ensure that it is fixed by clamping between the vertebral bodies (2).

5. Instrument set according to one of claims 1 to 4, characterized in that the guide surface (5) of the guide element (3) extends in the lateral direction.

6. Instrument set according to claim 5, characterized in that the guide surface (5) of the guide element (3) is a groove, and the guide surface (9a) of the working instrument (10) is formed by a guide mandrel (9) matching the width of the groove.

7. Instrument set according to claim 6, characterized in that the working instrument is a cylindrical milling cutter (10), and the guide mandrel (9) is rotatable relative to the guide surface (5) of the guide element (3).

8. Instrument set according to claim 5, characterized in that the working instrument (10) is a hand tool, for example a curette or rasp.

9. Instrument set according to one of claims 1 to 4, characterized in that the guide surface (5) of the guide element (3) is arranged centrally between the vertebral bodies (2) for simultaneous working of both vertebral bodies (2).

10. Instrument set according to one of claims 1 to 4, characterized in that it comprises an insertion element (4) for inserting the guide element (3).

11. Instrument set according to claim 10, characterized in that the insertion element (4) can be connected to the guide element (3) in a releasable manner.

12. Instrument set according to one of claims 2 to 4, characterized in that projections (7) for mechanical engagement in the vertebral body surface are arranged on the positioning surfaces.

13. Method for working a cervical vertebral body in order to receive an intervertebral joint prosthesis in the intervertebral space, characterized in that a guide element is positioned in the intervertebral space and has a guide surface lying inside said intervertebral space, and in that the vertebral body is worked by means of a working instrument while the latter is guided on the guide surface.

14. Method according to claim 13, characterized in that the guide element is fixed by clamping between the vertebral bodies.

15. Method according to claim 13 or 14, characterized in that the working instrument is guided on the guide surface parallel to the surface which is to be generated by the working.

16. Method according to claim 13 or 14, characterized in that a cylindrical milling cutter is used as working instrument.

17. Method according to claim 16, characterized in that two vertebral bodies are worked simultaneously.

18. Method according to claim 13 or 14, characterized in that, in order to insert the guide element into the intervertebral space, an insertion element is connected to the guide element in a releasable manner.
